(19) United States
(12) Patent Application Publication
(10) Pub. No.: US 2026/0345534 A1
(43) Pub. Date: Nov. 5, 2026

(54) DEUTERATED UREA FOR THE TREATMENT OF HYPONATREMIA

(71) Applicant: NEPHCENTRIC, LLC, Phoenix, AZ (US)

(72) Inventors: Bhupinder Singh, Phoenix, AZ (US); Lee Souders, Albuquerque, NM (US)

(21) Appl. No.: 19/656,010

(22) Filed: Apr. 23, 2026

Related U.S. Application Data
(60) Provisional application No. 63/793,435, filed on Apr. 23, 2025.

(51) Int. Cl. A61K 31/17

(52) U.S. Cl. CPC A61K 31/17

(57) ABSTRACT

Methods of treatment of hyponatremia using deuterated urea are provided.

### BACKGROUND

Hyponatremia is an electrolyte abnormality with a potential for significant morbidity and mortality. The condition arises when a person exhibits a positive balance of water with or without a deficit of sodium, which is recognized when the plasma sodium falls below the level of 135 mmol/L. Hyponatremia can occur in isolation in individuals who over-consume water. But more frequently hyponatremia results from a complication of medication or other underlying medical conditions leading to a diminished excretion of water.

Hyponatremia may lead to water intoxication, which results from retention of excess water causing the normal tonicity of extracellular fluid to fall below the safe limit. This may result in a potentially fatal disturbance in brain function. Typical symptoms of water intoxication include nausea, vomiting, headache, and malaise. Because nausea may also release anti-diuretic hormone (ADH), which causes water retention, this may cause a positive feedback loop that further worsens the symptoms of hyponatremia. Upon worsening conditions, a patient may experience reduced reflexes, convulsions, stupor, coma, or even death.

One way of treating hyponatremia is to administer urea to the patient. See, for example, 2014 European Society of Endocrinology, European Society of Intensive Care, European renal Association-EDTA. While the benefits of oral administration of urea are well documented, challenges exist in the development of an oral urea product that is acceptable for patients. Problems include delivering a clinically beneficial amount of urea and the undesirable organoleptic properties of urea compositions.

The present inventors have sought more effective compounds and compositions for the treatment hyponatremia.

### SUMMARY OF THE INVENTION

The present invention involves the treatment of hyponatremia by administering a therapeutically effective amount of deuterated urea to a patient in need thereof.

### DETAILED DESCRIPTION

Urea-d4 (1,1,3,3-Tetradeuteriourea) is a stable isotope of urea that is typically used for NMR analysis and is defined by the following Formula (I):

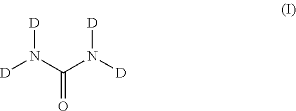

One way in which deuteration has been sought to improve the metabolic effects of existing pharmaceutical compounds is by slowing metabolism of the compound. This is often referred to as the kinetic isotope effect, whereby deuterium slows the metabolism of a particular compound by reducing the rate at which various decomposition steps occur. Urea, however, is excreted without metabolic degradation. Therefore, one would not expect any improvements in urea metabolism by way of deuteration.

The present inventors have observed that deuteration of urea has the potential to benefit the efficacy of urea in the treatment of hyponatremia for other reasons. One of the mechanisms of treatment of hyponatremia is the creation of an osmotic gradient across the blood-brain barrier. This gradient is reduced over time after urea diffuses through the blood-brain barrier. The present inventors believe that deuterated urea may diffuse through the blood-brain barrier at a slower rate than urea. In addition, urea is responsible for transport and excretion of water through the patient's kidneys. The deuterated urea may be more effective in transporting and excretion of water.

### Example 1

The following ingredients were added mixer to produce a dry powder, which was then added in a dose of 15 grams to a pouch.

| | | |
|---|---|---|
| Component | Amount | wt % |

| | | | |
|---|---|---|---|
| USP Urea-d4 | 15000 | mg | 67.0% |
| Sucralose | 210 | mg | 0.9% |
| Citric acid (anhydrous) | 1600 | mg | 7.1% |
| Natural Lemon-Lime Flavor | 2300 | mg | 10.3% |
| Maltodextrine M-500 | 2170 | mg | 9.7% |
| Calcium Silicate | 1120 | mg | 5.0% |

(Hubersorb ZeoFree 600)

The composition is prepared by adding into a mixer the urea-d4, maltodextrin, and calcium silicate. The calcium silicate is Hubersorb ZeoFree 600, which has a mean particle size of 8 microns, pH 10, BET Surface Area of 250 m²/g, and bulk density of 96 kg/m³. The resulting powder is free flowing a resistant to clumping. The powder composition is preferably packaged in a pouch that can be sprinkled onto food, or mixed with a liquid such as water. The composition forms a suspension when mixed with water, which allows for convenient ingestion. In this sense, the powder can be distributed and sold as a medical food. Alternatively, the powder may be incorporated into another medical food product such as a cookie or bar form similar to a diet bar.

### Example 2

The composition of Example 1 in a single-use sachet package is mixed with water and administered to a patient diagnosed with hyponatremia.

### Example 3

The following ingredients were added to a mixer to produce a dry powder:

| | | |
|---|---|---|
| Component | Amount | wt % |

| | | | |
|---|---|---|---|
| USP Urea-d4 | 15000 | mg | 67.0% |
| Calcium Silicate | 50 | mg | 0.3% |
| (particle size 3-7 micron) | | | |
| Silica (Sipernat 50S) | 250 | mg | 1.6% |

### Example 4

The composition of Example 3 taken from a resealable container is mixed with water and administered to a patient diagnosed with hyponatremia.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A method of treatment of hyponatremia comprising administering a therapeutically effective amount of a compound of Formula (I): 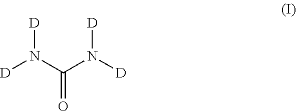
to a patient in need thereof.

2. The method of claim 1, wherein the compound of Formula (I) is present in a power composition comprising:
(a) 60-80 wt % of the compound of Formula (I);
(b) 7-12 wt % maltodextrin; and
(c) 5 wt % calcium silicate, wherein the powder composition is effective for treatment of hyponatremia.

3. The method of claim 2, wherein the composition comprises about 9.7 wt % maltodextrin.

4. The method of claim 2, further comprising a flavoring agent.

5. The method of claim 2, further comprising citric acid, sucralose, and a flavoring agent.

6. The method of claim 2, wherein the composition is administered by dispersing or dissolving the composition in water prior to consumption.

7. The method of claim 2, wherein the composition is administered by mixing the composition with food prior to consumption.

8. The method of claim 1, wherein the compound of Formula (I) is present in a power composition comprising:
(a) an amount of the compound of Formula (I);
(b) silica; and
(c) calcium silicate.

9. The method of claim 8, wherein the calcium silicate has a particle size in the range of 3 to 7 microns, wherein less than 10% of the particles by weight have a diameter less than 3 microns or greater than 7 microns.

10. The method of claim 8, the silica having a mean particle size in the range of 10-25 microns.

11. The method of claim 8, wherein the composition comprises 95-99.5 wt % urea.

12. The method of claim 8, wherein the composition comprises 0.05-1 wt % calcium silicate.

13. The method of claim 8, wherein the composition comprises 0.5-5 wt % silica

14. The method of claim 8, wherein the composition lacks a flavoring agent.

15. The method of claim 8, wherein the composition is administered from a resealable container.
